(19) United States
(12) Patent Application Publication
(10) Pub. No.: US 2024/0350304 A1
(43) Pub. Date: Oct. 24, 2024

(54) EXOTHERMIC ADHESIVE NOSE STRIP

(71) Applicants: Babak Ghalili, New York, NY (US); Emma Ghalili, New York, NY (US); John Borja, Keyport, NJ (US)

(72) Inventors: Babak Ghalili, New York, NY (US); Emma Ghalili, New York, NY (US); John Borja, Keyport, NJ (US)

(21) Appl. No.: 18/636,908

(22) Filed: Apr. 16, 2024

Related U.S. Application Data
(60) Provisional application No. 63/496,731, filed on Apr. 18, 2023.

(51) Int. Cl. A61F 7/03; A61F 7/00; A61F 7/02

(52) U.S. Cl. CPC A61F 7/034; A61F 2007/0006; A61F 2007/0204; A61F 2007/0226; A61F 2007/0244; A61F 2007/038

(57) ABSTRACT

The present disclosure relates to exothermic nose strip devices and compositions, including, methods of making and using them.

### FIELD

The aspects of the present disclosure relate to an exothermic adhesive nose strip including active agents used therein, methods of making and methods of using it.

### BACKGROUND

Nasal strips have been used by a segment of the human population that has some malformation of the nasal passages that interferes with breathing, including deviated septa or inflammation due to infection or allergic reactions. For this use, part of the interior nasal passage wall may draw in during inhalation to substantially block the flow of air. Blockage of the nasal passages as a result of malformation, or nasal congestion symptoms of the common cold or seasonal allergies are particularly uncomfortable at night, and can lead to sleep disturbances, irregularities and general discomfort.

### SUMMARY

In one embodiment, a nose strip device is provided. The nose strip device includes first and second sections that are discrete and space apart from one another both including an exothermic material contained therein and an adhesive substance disposed on at least a portion of the first and second sections for engaging the nose strip device to a nose of a user and a bridge having first and second end portions, the first end portion bonded to at least a portion of the first section, the second end portion bonded to at least a portion of the second section.

In another embodiment, a method of warming the tissues of a nose of a user is provided. The method includes using a nose strip device including first and second sections that are discrete and space apart from one another both including an exothermic material contained therein and an adhesive substance disposed on at least a portion of the first and second sections for engaging the nose strip device to the nose of the user and a bridge having first and second end portions, the first end portion bonded to at least a portion of the first section, the second end portion bonded to at least a portion of the second section; activating the exothermic material to give off heat; and applying the nose strip device to the nose of the user.

In another embodiment, a method of warming the tissues of a nose of a user to maintain normal exosome production is provided. The method includes using a nose strip device including first and second sections that are discrete and space apart from one another both including an exothermic material contained therein and an adhesive substance disposed on at least a portion of the first and second sections for engaging the nose strip device to the nose of the user and a bridge having first and second end portions, the first end portion bonded to at least a portion of the first section, the second end portion bonded to at least a portion of the second section; activating the exothermic material to give off heat; and applying the nose strip device to the nose of the user to warm the tissues of the nose in order to maintain normal exosome production.

### BRIEF DESCRIPTION OF THE DRAWINGS

The accompanying drawings illustrate presently preferred embodiments of the present disclosure, and together with the general description given above and the detailed description given below, serve to explain the principles of the present disclosure.

FIG. 1 is a cross-sectional view of an embodiment of the present disclosure;

FIGS. 2A, 2B and 2C are perspective views of tabletized exothermic material for embodiments of the present disclosure;

FIG. 3 is a top view showing dimensions of one embodiment of the present disclosure; and

FIGS. 4A and 4B are front and side views of a human face with an embodiment of the present disclosure.

### DETAILED DESCRIPTION

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s).

The use of the terms “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by embodiments of the present disclosure. As used herein, “about” may be understood by persons of ordinary skill in the art and can vary to some extent depending upon the context in which it is used. If there are uses of the term which are not clear to persons of ordinary skill in the art, given the context in which it is used, “about” may mean up to plus or minus 10% of the particular term.

The terms “%”, “% by weight”, “weight %” and “wt %” are all intended to mean unless otherwise stated, percents by weight based upon a total weight of 100% end composition weight. Thus 10% by weight means that the component constitutes 10 wt. parts out of every 100 wt. parts of total composition.

The aspects of the disclosed embodiments relate to a nose strip device including a porous film/pad/fabric/nonwoven which contains chemicals which can generate an exothermic, for example, when exposed to air, including, for example, iron in powder form. The aspects of the disclosed embodiments also relate to processes for the preparation of the nose strip and the uses of such a nose strip that warms the nose exothermically, especially during the winter months. Studies have shown that if the inner nose drops 9° F., then exosome production is decreased by about half, resulting in resulting in an immune response that is not as strong.

One specific embodiment of the present disclosure relates to a nose strip device comprising an exothermic active agent including, for example, iron that will exotherm due to oxidation when exposed to air. Other metal salts that are prone to oxidation can also be used such as compounds of copper, alkaline sulfides, lithium, potassium, calcium, sodium, magnesium, aluminum, zinc, chromium, cobalt, nickel, lead, iron carbides etc.

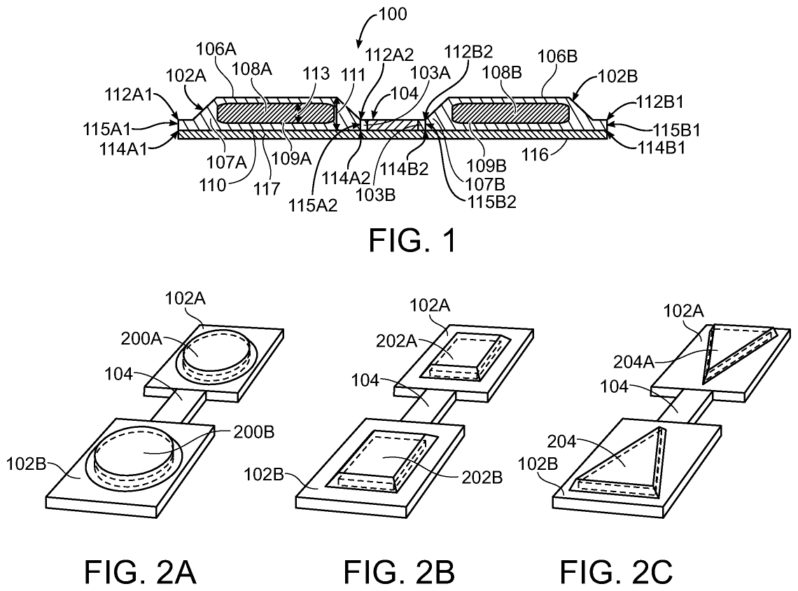

One embodiment of the present disclosure is illustrated in FIG. 1. FIG. 1 embodiment 101 includes two discrete sections 102A and 102B that are spaced apart and connected by a nose bridge 104 at 103A and 103B, respectively. The nose bridge 104 can be flexible/bendable/resilient. Sections 102A and 102B can be of the same or different in composition, structure, size and shape. Each section 102A and 102B includes a porous film/semi-porous/pad/fabric/nonwoven material 106A and 106B where at least one of sections 102A and 102B, usually both, includes a cavity 107A and 107B respectively and a portion of tabletized or freely moveable exothermic material 108A and 108B within at least one of cavities 107A and 107B usually both cavities 107A and 107B. The exothermic material 108A and 108B can be the same or different and can produce exothermic heat that can range from about 100° F. to about 140° F., that can be the same or different between exothermic material 108A and 108B. The thickness 111 of cavity 107A can range from about 1.5 mm to about 3.0 mm and where the thickness of 107B can be substantially the same as thickness 111. The tabletized or freely moveable exothermic material 108A and 108B can be, but not necessarily, positioned within a flexible or nonflexible envelope 109A and 109B. The thickness 113 of tabletized or freely moveable exothermic material 108A can range from about 1.5 mm to about 2.5 mm where the thickness of 108B can be substantially the same as thickness 113. A section of non-porous film 110 with an adhesive substance 117 is facing outwardly on the surface of the non-porous fabric/film 114A and 114B as shown in FIG. 1. End portions 112A1, 112A2, 112B1 and 112B2 of each section of the porous film/semi-porous/pad/fabric/nonwoven material at 106A and 106B respectively and end portions 114A1, 114A2, 114B1 and 114B2 of non-porous fabric/film 110 can be heat-sealed at their opposing end portions at 115A1, 115A2, 115B1 and 115B2. A peel off release film 116, for example, wax paper can be attached to the section of non-porous film 110. The peel off release film 116 is removed to expose the adhesive substance 117 of the non-porous fabric/film 110 when the nose strip device is to be used and attached to the nose. The nose bridge is flexible/bendable/resilient so that the non-porous film 110 of sections 102A and 102B can both be in contact with the external tissue of the nose.

The porous film/semi-porous/pad/fabric/nonwoven material 106A and 106B can include, for example, blends of polyester, rayon, cotton, polyethylene, polypropylene, paper, spunbound, spunlace, melt blown, staple fibers, in particular spunbound polypropylene nonwoven with a pore size ranging from about 3 and about 20 microns, about 10 microns. The thickness of semi-porous permeable nonwoven fabric/film 106A and 106B can range from about 0.10 mm to about 0.30 mm, about 0.15 mm.

The non-porous fabric/film 110 can be made of blends of metal foil, PET/polyester, polyimide, PVC/Vinyl, Silicone, Acrylics, PTFE's, LDPE/HDPE, Polypropylene/ethylene, non-porous spunbound polypropylene, spunbound, spunlace, melt blown, staple fibers. The adhesive substance 117 used with the non-porous fabric/film can include topical pressure sensitive skin adhesives or self-stick adhesives such as, for example, acrylics, epoxies, hydrocolloids, hydrogels, rubber based thermoplastics, polyurethanes silicones, cyanoacrylates as well as a pressure sensitive topically acceptable adhesive, such as, for example, acrylics, butyl rubber, ethylene-vinyl acetate (EVA), natural rubber, nitriles, silicone rubbers, styrene block copolymers (SBC), styrene-butadiene-styrene (SBS), styrene-ethylene/butylene-styrene (SEBS), styrene-ethylene/propylene (SEP), styrene-isoprene-styrene (SIS), as well as hydrophilic pressure sensitive adhesives (PSA) such as for example polyvinylpyrrolidone-polyethylene glycol copolymers (PVP-PEG), maleic anhydride vinyl ether-methyl- and amine-terminated polyethylene glycol copolymers (MVE/MA-PEG), and polyethylene oxide-polyethylene glycol copolymers (PEO-PEG), particularly acrylic skin adhesives.

Peel off release film 116 can be made of, for example, paper (such as, for example, craft paper, wax paper and glassine, clay coated, PVOH coated, glazed coated, silicone coated papers) or plastic (such as, for example, PET, polypropylene, polyolefins such as LDPE/HDPE), particularly silicone coated release paper.

Nose bridge 104 material should be flexible or bendable to maintain its shape once bent or resilient to be bendable and to return to its original shape and can be made, for example, of fabric made (such as, for example, polyester, rayon, cotton, paper, etc.), plastic (such as, for example, PET, polypropylene, polyolefins such as LDPE/HDPE) or metals (such as, for example, aluminum, etc. that can bend to the shape of bridge of nose), particularly non-porous spunbound polypropylene nonwoven.

FIGS. 2A, 2B and 2C include embodiments that include different geometric shapes of tabletized and/or or freely moveable exothermic material (with or without an envelope), 200A and 200B in FIG. 2A, 202A and 202B in FIGS. 2B and 204A and 204B in FIG. 2C that each may of the approximately same or different geometric shapes all of which include sections 102A and 102B and nose bridge 104 as detailed in FIG. 1.

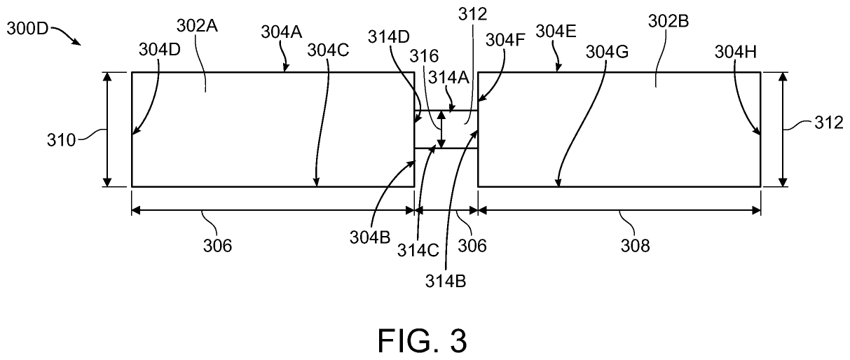

FIG. 3 is an embodiment of the present disclosure that can be similar or the same as the embodiment illustrated in FIG. 1 and illustrates the external dimensions of an embodiment of the present disclosure. Embodiment 300 of FIG. 3 includes sections 302A and 302B. Section 302A includes sides 304A, 304B, 304C and 304D. Section 302B includes sides 304E, 304F, 304G and 304H. Sides 304A and 304C can be about the same or different and range in length 306 for adults about 2.5 cm to about 3.5 cm, about 3.0 cm and for children (ages from about 5 years of age to about 12 years of age) about 2.0 cm to about 3.0 cm, about 2.5 cm. Length 308 of sides 304E and 304G can be about the same or different from each other and about the same or different as length 306 for sides 304A and 304C and length 308 has the same ranges for adults and children included for length 306. Sides 304B and 304D can be about the same or different and range in width 310 for adults about 2.0 cm to about 3.0 cm, about 2.5 cm and for children (ages from about 5 years of age to about 12 years of age) about 1.0 cm to about 2.0 cm, about 1.5 cm. Width 310 of sides 304B and 304D can be about the same or different from each other and about the same or different as width 312 for sides 304F and 304H and width 312 has the same size ranges for adults and children included for width 310.

Nose bridge 312 includes sides 314A, 314B, 314C and 314D. Sides 314A and 314C can be about the same or different and range in length 306 from about 1.0 cm to about 2.0 cm, about 1.5 cm. Length 306 of sides 314A and 314C can be about the same or different from each other. Sides 314B and 314D can be about the same or different and range in width 316 from about 0.5 cm to about 1.5 cm, about 1.0 cm.

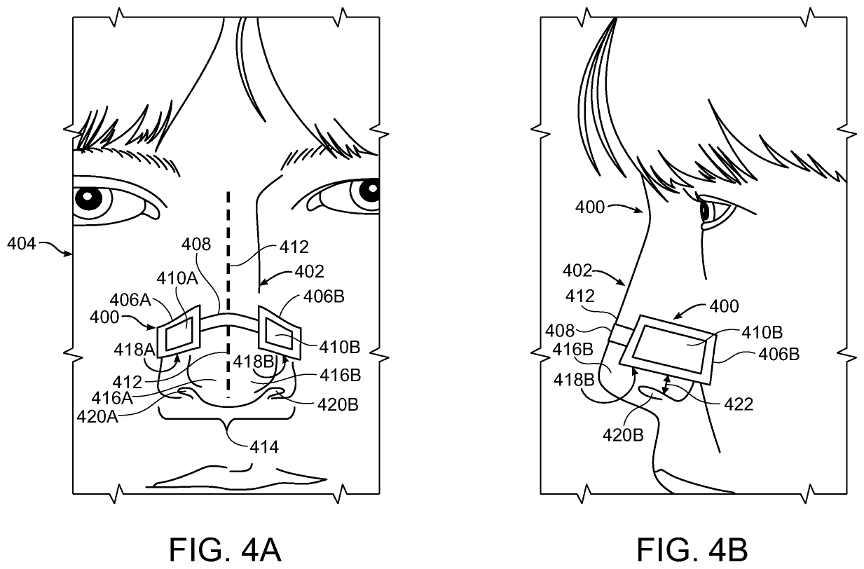

FIGS. 4A and 4B illustrate nose strip device embodiments of the present disclosure, such as, for example, the nose strip embodiment illustrated in FIG. 1 and illustrates the nose strip device embodiments when applied to the nose of a human user. In FIGS. 4A and 4B, the nose strip device 400 has been applied to the nose 402 of human user 404. The nose strip device 400 includes two sections 406A and 406B that are connected by a nose bridge 408, sections 406A and 406B that include exothermic material 410A and 410B, respectively, similar to those that are exemplified in FIG. 1. Bridge 408 is positioned on nose 402 to cover ridge 412 thereof and such that sections 406A and 406B are positioned toward the nostril side 414 and are attached to opposing sides 416A and 416B of nose 402 and the exterior nasal tissue thereof using adhesive sides 418A and 418B on the underside of each section. Section 406A is proximal to or adjacent to nostril 420A and section 406B is proximal to or adjacent to nostril 420B and warmth of the exothermic material 410A and 410B is applied to sides 416A and 416B of nose 402. In an embodiment the distance 422 section 406B is from nostril 420B can be from about 0.5 cm to about 1.5 cm and the distance section 406A is from nostril 420A is approximately the same.

Exothermic ingredients can include, for example, alkali metals and its compounds (Li, Na, K, Rb, Cs, and Fr), alkaline earth metals and its compounds (Be, Mg, Ca, Sr, Ba, and Ra), halogens and its compounds (F, Br, I, Cl) and exothermic clays (zeolite, metakaolinite, etc.). The exothermic ingredients become activated and give off heat upon exposure to moisture (e.g., in the air and/or upon coming in contact with a body part such as, for example, a hand or the nose).

Additional ingredients that can be used in the exothermic materials of embodiments of the present disclosure include activated charcoal or acceptable alternatives, sodium chloride, vermiculite, water and hypromellose.

Activated charcoal is thermally synergistic, holds water, helps spread heat evenly. Additionally, alternates to charcoal can include clays such as, for example, laponite, zeolite, kaolin, hectorite, stevensite, kerolite, smectite, etc.

Sodium chloride is hygroscopic and is the catalyst for iron oxidation to occur. Other chlorides can include, for example, potassium, calcium, magnesium and zinc chloride salts.

Vermiculite, as with activated charcoal, vermiculite helps spread heat evenly and as a filler. It also allows air to penetrate the mixture. Other fillers can include, for example, talc, microcrystalline cellulose, starches, powdered wood etc.

Water as with sodium chloride, water is also a catalyst to start the exothermic oxidation of iron.

Hypromellose is a binder that holds the powder mixture together. Other binders can include, for example, PVP, HPMC, pullulan, sodium CMC, sodium alginate, polyvinyl alcohol, etc.

Another embodiment may include the results of the process of mixing the powder with different exothermic material. The process includes premixing water with activated charcoal. Then vermiculite, sodium chloride, and hypromellose are dry mixed together. Iron is then added but has to be admixed in an oxygen-free environment. Inert gases such as argon, and/or nitrogen can be used to fill the headspace during mixing displacing oxygen. The powder is then tabletized to form a rectangular, oval, or triangular shapes. This “tablet” is then sandwiched or pouched within the cavities of a nose strip (for example, as shown in FIG. 1) and is packaged in an air and moisture impermeable sealed oxygen free container or envelope to form, for example, a kit. Examples of air and moisture impermeable material that can be used include, for example, foil laminate, polyethylene films, polyester films, polyethylene furanoate films, nylon films, polylactic acid films, PVC films, cellophane films, etc.

Another embodiment includes methods of using, applying warmth, warming the tissues of the nose of a user (e.g., a human user) including the nose strip embodiments of the present disclosure. As exemplified in FIGS. 4A and 4B, the use includes applying the nose strip 400 where sections 406A and 406B including exothermic material 410A and 410B are positioned on opposing sides of the nose 402 and adhering it to the nose 402 using the adhesive sides 418A and 418B of sections 406A and 406B, respectively. The nose bridge 104 is placed in contact with the ridge 412 of nose 402 to which the nose strip is attached. Embodiments of the present disclosure also include applying warmth or warming to the nose and external and internal tissues thereof via the exothermic material, for example, to maintain the normal body temperature thereof (the normal body temperature being from about 97° F. to about 99° F. where the nose is exposed to colder than room temperature (room temperature being from about 68° F. to about 74° F.), in order to maintain exosome production at a normal rate resulting in a normal immune response. The immune response being the human body's ability to recognize the antigens (usually proteins) on the surface of substances or microorganisms, such as, for example, bacteria or viruses, and attack and destroy, or try to destroy, them.

Embodiments of the present disclosure also include applying warmth or warming to the nose thereof via the exothermic material, for example, to maintain the normal body temperature thereof (the normal body temperature being from about 97° F. to about 99° F. where the nose is exposed to colder than room temperature (room temperature being from about 68° F. to about 74° F.), in order for cells in the front of the nose to maintain at a normal rate the detection of infections (including, for example, bacteria and/or viruses) and then release fluid-filled sacs called extracellular vesicles (or EVs, known previously as exosomes) into the mucus to surround and attack the infection.

Embodiments of the present disclosure also include applying warmth or warming to the nose thereof via the exothermic material, for example, to maintain the normal body temperature thereof (the normal body temperature being from about 97° F. to about 99° F. where the nose is exposed to colder than room temperature (room temperature being from about 68° F. to about 74° F.), in order to maintain the normal secretion of nasal epithelial EVs against infections via Toll-like receptor 3 (TLR3) signaling, including, for example, viral infections, for example, via Toll-like receptor 3 (TLR3) signaling and the activity, for example, antiviral activity, and mechanisms of TLR3-stimulated EVs against infections (including, for example, respiratory infections), including, for example, viral infections.

In a further embodiment, a kit is disclosed. One example of such a kit is a kit including one or more of the embodiments of the present disclosure separately packaged and instructions for use.

### Example 1

| | Exothermic heating | | | | |
|---|---|---|---|---|---|
| Item# | Ingredients | Function | w/w % | Lower | Upper |
| 1 | Iron powder/filings | | 53.0 | 43.0 | 63.0 |
| 2 | Activated charcoal | | 10.0 | 5.0 | 15.0 |
| 3 | Sodium chloride | | 13.2 | 8.0 | 18.0 |
| 4 | Hypromellose | | 0.7 | 0.2 | 1.2 |
| 5 | Vermiculite | | 13.1 | 8.0 | 18.0 |
| 7 | Water | | 10.0 | 5.0 | 15.0 |
| | | TOTAL | .0 | | |

This written description uses examples as part of the disclosure, including the best mode, and also to enable any person skilled in the art to practice the disclosed implementations, including making and using any devices or systems and performing any incorporated methods. The patentable scope is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

While there have been shown, described and pointed out, fundamental features of the present disclosure as applied to the exemplary embodiments thereof, it will be understood that various omissions and substitutions and changes in the form and details of compositions, devices and methods illustrated, and in their operation, may be made by those skilled in the art without departing from the spirit or scope of the present disclosure. Moreover, it is expressly intended that all combinations of those elements and/or method steps, which perform substantially the same function in substantially the same way to achieve the same results, are within the scope of the present disclosure. Moreover, it should be recognized that structures and/or elements and/or method steps shown and/or described in connection with any disclosed form or embodiment of the present disclosure may be incorporated in any other disclosed or described or suggested form or embodiment as a general matter of design choice. It is the intention, therefore, to be limited only as indicated by the scope of the claims appended hereto.

1. A nose strip device, comprising:
first and second sections that are discrete and space apart from one another both including an exothermic material contained therein and an adhesive substance disposed on at least a portion of the first and second sections for engaging the nose strip device to a nose of a user; and
a bridge having first and second end portions, the first end portion bonded to at least a portion of the first section, the second end portion bonded to at least a portion of the second section.

2. The nose strip device according to claim 1, wherein the first and second sections each further include a first layer and a second layer with at least one cavity positioned there between and the exothermic material disposed in the at least one cavity.

3. The nose strip device according to claim 2, wherein the first layer of the first and second sections includes a porous film/semi-porous/pad/fabric/nonwoven material, the second layer of the first and second sections includes non-porous fabric/film, the second layer having an external surface on which is disposed the adhesive substance.

4. The nose strip device according to claim 1, wherein the exothermic substance is positioned in an envelope that is flexible or non-flexible.

5. The nose strip device according to claim 1, wherein the exothermic substance is tabletized or freely moveable.

6. A method of warming the tissues of a nose of a user, comprising:
using a nose strip device including:
first and second sections that are discrete and space apart from one another both including an exothermic material contained therein and an adhesive substance disposed on at least a portion of the first and second sections for engaging the nose strip device to the nose of the user; and
a bridge having first and second end portions, the first end portion bonded to at least a portion of the first section, the second end portion bonded to at least a portion of the second section;
activating the exothermic material to give off heat; and
applying the nose strip device to the nose of the user.

7. The method according to claim 6, wherein the first and second sections each further include a first layer and a second layer with at least one cavity positioned there between and the exothermic material disposed in the at least one cavity.

8. The method according to claim 7, wherein the first layer of the first and second sections includes a porous film/semi-porous/pad/fabric/nonwoven material, the second layer of the first and second sections includes non-porous fabric/film, the second layer having an external surface on which is disposed the adhesive substance.

9. The method according to claim 6, wherein the exothermic substance is positioned in an envelope that is flexible or non-flexible.

10. The method according to claim 6, wherein the exothermic substance is tabletized or freely moveable.

11. The method according to claim 6, wherein the exothermic material maintains the normal body temperature of the nose where the nose is exposed to colder than room temperature.

12. The method according to claim 6, further including applying the first section of the nose strip device to one side of the nose and applying the second section of the nose strip device to the other side of the nose to warm the tissues of the nose wherein the distance of the first and second sections of the nose strip device from an adjacent nostril thereto is from about 0.5 cm to about 1.5 cm.

13. A method of warming the tissues of a nose of a user to maintain normal exosome production, comprising:
using a nose strip device including:
first and second sections that are discrete and space apart from one another both including an exothermic material contained therein and an adhesive substance disposed on at least a portion of the first and second sections for engaging the nose strip device to the nose of the user; and
a bridge having first and second end portions, the first end portion bonded to at least a portion of the first section, the second end portion bonded to at least a portion of the second section;
activating the exothermic material to give off heat; and
applying the nose strip device to the nose of the user to warm the tissues of the nose in order to maintain normal exosome production.

14. The method according to claim 13, wherein the first and second sections each further include a first layer and a second layer with at least one cavity positioned there between and the exothermic material disposed in the at least one cavity.

15. The method according to claim 14, wherein the first layer of the first and second sections includes a porous film/semi-porous/pad/fabric/nonwoven material, the second layer of the first and second sections includes non-porous fabric/film, the second layer having an external surface on which is disposed the adhesive substance.

16. The method according to claim 13, wherein the exothermic substance is positioned in an envelope that is flexible or non-flexible.

17. The method according to claim 13, wherein the exothermic substance is tabletized or freely moveable.

18. The method according to claim 13, wherein the exothermic material maintains the normal body temperature of the nose where the nose is exposed to colder than room temperature.

19. The method according to claim 13, further including applying the first section of the nose strip device to one side of the nose and applying the second section of the nose strip device to the other side of the nose such that the distance of the first and second sections of the nose strip device from an adjacent nostril of the nose is from about 0.5 cm to about 1.5 cm.

20. The method according to claim 13, further including maintaining the normal secretion of exosomes against infections.
